(19) United States
(12) Patent Application Publication
(10) Pub. No.: US 2010/0305458 A1
(43) Pub. Date: Dec. 2, 2010

(54) BLOOD PRESSURE METER AND METHOD FOR OPERATING A BLOOD PRESSURE METER

(76) Inventors: Ulrich Pfeiffer, Munich (DE); Reinhold Knoll, Munich (DE); Matthias Faehle, Munich (DE)

Correspondence Address: Leydig, Voit & Mayer, Ltd. (Frankfurt office), Two Prudential Plaza, Suite 4900, 180 North Stetson Avenue, Chicago, IL, 60601-6731, US

(21) Appl. No.: 12/682,713

(22) PCT Filed: Oct. 8, 2008
(86) PCT No.: PCT/EP08/08506

§ 371 Date: Aug. 6, 2010

(30) Foreign Application Priority Data
Oct. 11, 2007 (DE) 10 2007 048 880.9

(51) Int. Cl. A61B 5/0215

(52) U.S. Cl. 600/486

(57) ABSTRACT

A blood-pressure measuring device includes a resistor having a first end and a second end, a first pressure sensor disposed on the first end and a second pressure sensor disposed on the second side and configured for connection to a blood circuit, wherein the first pressure sensor, the resistor and the second pressure sensor are connected to each other in series. A computing device is connectable to the first pressure sensor and the second pressure sensor and is configured to perform a plausibility check by comparing measurement results of the first pressure sensor and of the second pressure sensor.

### CROSS-REFERENCE TO PRIOR APPLICATIONS

This application is a U.S. National Phase application under 35 U.S.C. §371 fo International Application No. PCT/EP2008/008506, filed on Oct. 8, 2008 and claims priority to German Patent Application No. DE 10 2007 048 880.9, filed on Oct. 11, 2007.

The present invention relates to a blood-pressure measuring device and method for operating a blood-pressure measuring device

### BACKGROUND

Direct blood-pressure measurement is known in the state of the art. Blood-pressure measurement is carried out by means of a pressure transducer in a blood vessel.

In US 2006/0009699 a blood-pressure measuring apparatus is described which has two pressure sensors, in order to provide two separate but identical output values.

In the case where, while blood pressure is being measured, access routes via a catheter were blocked for example, or measurement values were distorted by kinks in the catheter in the bloodstream, the blood-pressure measuring devices of the state of the art display a false value.

### SUMMARY

In an embodiment, the present invention provides, a blood-pressure measuring device that includes a resistor having a first end and a second end, a first pressure sensor disposed on the first end of the resistor and a second pressure sensor disposed on the second side of the resistor and configured for connection to a blood circuit. The first pressure sensor, the resistor and the second pressure sensor are connected to each other in series. A computing device connectable to the first pressure sensor and the second pressure sensor is configured to perform a plausibility check by comparing measurement results of the first pressure sensor and of the second pressure sensor.

### BRIEF DESCRIPTION OF THE DRAWINGS

Preferred embodiments of the invention are described in more detail below using the attached drawings. There are shown in:

FIG. 1 a diagram of an embodiment example of a blood-pressure measuring device according to the invention;

FIG. 2 a further diagram of an embodiment example of a blood-pressure measuring device according to the invention and

FIG. 3 a flowchart illustrating the course of a method according to the invention.

### DETAILED DESCRIPTION

In an embodiment the present invention provides a device and a method with which a more reliable blood-pressure measurement is made possible.

In an embodiment, a blood-pressure measuring device includes a flow resistor, a first pressure sensor provided on a first side of the resistor and set up for connection to a rinsing device, a second pressure sensor provided on the second side of the resistor and set up for connection to a blood circuit, wherein the first pressure sensor, the resistor and the second pressure sensor are connected in series and the first pressure sensor and the second pressure sensor can be connected to a computing device, by which, by comparing measurement results of the first pressure sensor and of the second pressure sensor, a plausibility check can be carried out.

Preferably, blood pressure is measured outside the body by a pressure sensor via a liquid-filled catheter. In order to prevent blood clots forming at the tip of the catheter and the catheter narrowing or closing as a consequence of activation of the coagulation system by the foreign surfaces of the catheter material, the pressure-measurement lumen of the catheter is preferably constantly rinsed with a small quantity of liquid. Preferably, isotonic 0.9% common salt solution is used as liquid. Preferably, the rinsing rate is 3 ml per hour. It is possible, through the blood-pressure measuring device according to the invention, to check the rinsing of the catheter.

The blood pressure is preferably the pressure of a pulsed wave in the blood which is acting in the blood vessels. Preferably, the blood pressure is measured as relative pressure. Preferably, the blood pressure is measured in a large artery. Preferably, the blood pressure is measured by means of a pressure transducer in a liquid-filled tube. It is thereby possible to obtain an accurate value for the blood pressure.

A flow resistor is preferably a resistor which creates in a gaseous and/or liquid medium a force acting against the movement direction. Preferably, by providing the resistor, different flow properties are created in the gaseous and/or liquid medium in front of and behind the resistor. The resistor is particularly preferably suitable for creating different pressures in front of and behind the resistor. Preferably, the resistor is designed as a rinsing capillary. A capillary is preferably a very fine, elongated cavity. It is thereby possible to create a laminar flow. This has the advantage that properties of this flow can be particularly well calculated and a capillary produced that can be very well reproduced.

Preferably, the resistor is designed as a diaphragm. A diaphragm is preferably a component with a narrow cross-section which is set up to have at least a gas and/or a liquid flow through it. It is thereby possible to design the resistor in particularly cost-favourable manner e.g. by injection moulding or lasers. A diaphragm is also less likely to become blocked and is empirically well calculable.

A pressure sensor is preferably a sensor which is suitable for measuring the static pressure in a gas and/or a liquid. Preferably, a pressure sensor is set up to convert the physical variable pressure into an electric output value proportional to the pressure.

Preferably, a piezoresistive pressure sensor is used. The blood-pressure measuring device can thereby be provided at favourable cost. Preferably, a piezoelectric pressure sensor is used. It is thereby possible to dispense with an external power supply. Moreover, natural oscillations and reverberation effects are minimized Preferably, strain gauges are used as pressure sensors. Any type of path measurement in combination with an elastic structure is also conceivable.

Preferably, the rinsing device is a bag with a drip controller (gravity infusion). Preferably, the bag is provided with a pressure cuff. Preferably, a pressure between 200 and 500 mmHg is created by the rinsing device. Particularly preferably a pressure between 290 and 310 mmHg is created by the rinsing device.

Preferably, the first pressure sensor is arranged outside the body of the patient. Preferably, the first pressure sensor, the resistor and the second pressure sensor are connected in series such that the pressure of a fluid in the first pressure sensor can be measured before the fluid has flowed through the resistor and the pressure of the fluid after it has flowed through the resistor is measured by the second pressure sensor.

Preferably, a computing device is an apparatus which can process information with the help of a programmable computational protocol. Preferably, a monitor is used as computing device. A monitor is preferably a device or combination of devices with which vital parameters of a living being can be measured and monitored. The use of an additional device is thereby avoided. Preferably, a microcontroller or a microprocessor is used. An independent device can thereby be provided which can be specially set up to carry out a plausibility check. Preferably, microcontrollers are provided as computing devices, in particular if there are further tasks to be carried out. A comparator or a coupling logic circuit is also conceivable.

A plausibility check is a check which is set up to examine whether measured values can occur in the system if it is functioning properly.

If there is no rinsing, the system does not function properly. This can occur if e.g. a reservoir for the rinsing liquid more particularly of the bag is empty, a cock which is provided between the rinsing device and the patient has not been opened, a tube provided between the rinsing device and the patient has a kink in it, a tip of a catheter via which the blood pressure is measured is placed unfavourably in the bloodstream or the catheter has a kink in it inside or outside the body. If there is no rinsing, deposits can increasingly narrow or completely block the catheter opening. An incorrect pressure which depends on the state of the blockage can then be measured at the second pressure sensor. Depending on the degree of the deposition, the measurement value can acquire any value between blood pressure and rinsing pressure. This situation cannot be recognized from the measurement signal, as a portion of the blood-pressure signal is also included in the measurement signal. Misdiagnoses are thereby possible. It is moreover possible that blood clots form which when detached either spontaneously or by thorough rinsing, for example manual rinsing at very high pressure, can lead to a downstream embolism.

The plausibility check preferably comprises a check as to whether the pressure at the first pressure sensor is close to a threshold value which corresponds to the minimum pressure in the rinsing liquid reservoir plus the water column on the pressure sensor. If this is the case an error in the rinsing liquid feed or an empty rinsing reservoir can be assumed.

The plausibility check preferably also comprises a check as to whether the difference in pressure between the measurement values of the first pressure sensor and the measurement values of the second pressure sensor is decreasing. If this is the case it can be concluded that the cross-section of a catheter has narrowed or closed completely between the second pressure sensor and the patient.

Preferably, the plausibility check comprises a check as to whether a predetermined difference in pressure between the measurement value of the pressure measured by the first pressure sensor and the pressure measured by the second pressure sensor fails to reach a predetermined value. Alternatively, a check as to whether the calculated flow fails to reach a predetermined value. In this case it can be concluded that an adequate rinsing with rinsing liquid is not guaranteed. This can be caused by a cock between the second pressure sensor and the patient being closed, a tube between the second pressure sensor and the patient being closed, the tip of the catheter lying unfavourably in the bloodstream, the presence a kink in the catheter inside and/or outside the body and/or deposits being present on the tip of the catheter.

With the blood-pressure measuring device according to the invention it is possible to monitor and ensure a continuous rinsing process and thereby avoid complications due to blood clots. Moreover, it is possible to avoid false interpretations of the pressure measurement.

Preferably, the plausibility check comprises a check,

as to whether the pressure p1 measured by the first pressure sensor is lower than a predetermined maximum pressure p1max and/or

a check as to whether the pressure p1 measured by the first pressure sensor is higher than a predetermined minimum pressure p1min and/or a check as to whether the difference between the pressure p1 measured by the first pressure sensor and the pressure p2 measured by the second pressure sensor is more than a predetermined minimum differential pressure DELTapmin and/or

a check as to whether the difference between the pressure p1 measured by the first pressure sensor and the pressure p2 measured by the second pressure sensor is less than a predetermined maximum differential pressure DELTapmax. It is thereby possible to easily determine whether the measured pressure values are plausible or whether there is a fault in the system. The predetermined maximum pressure p1max is preferably between 300 mmHg and 500 mmHg, particularly preferably 500 mm Hg.

This predetermined maximum pressure p1max could for example be exceeded by manual flush rinsing or incorrect operation.

The predetermined minimum pressure p1min is preferably between 200 and 300, particularly preferably 200 mm Hg.

A failure to reach the value p1min can indicate that the rinsing device or the rinsing liquid feed has been disrupted. If p1 falls below the value p1min it could be that the bag is empty. The predetermined minimum differential pressure DELTapmin is preferably between 20 and 100 mmHg, particularly preferably 50 mmHg If DELTapmin is not reached it can be concluded that too small a quantity of liquid is flowing through the resistor.

The predetermined maximum differential pressure DELTapmax is preferably between 400 mmHg and 600 mmHg, particularly preferably 500 mmHg The maximum differential pressure DELTapmax could for example be exceeded by manual flush rinsing or by incorrect operation.

Preferably, the blood-pressure measuring device also comprises an alarm device, wherein the alarm device is set up to sound an alarm when the maximum pressure or the maximum differential pressure is exceeded or when the minimum pressure or the minimum differential pressure is not reached. It is thereby possible to draw attention to a malfunctioning of the blood-pressure measuring device.

A specific target group, preferably doctors and/or care workers, is thus made aware of an incident by an alarm. Preferably, the target group reacts to the incident. An alarm device is a device which is suitable for drawing the attention of the target group to the incident. Preferably, a device is used which gives an audible signal. The target group can thereby also be reached if their attention is not directed towards the blood-pressure measuring device. Preferably, a device is used which gives a visual signal. It is thereby possible to reach a target group especially directly. It is also thereby easily possible to give further information.

Preferably, the blood-pressure measuring device also comprises a display device, wherein the display device is set up to display the result of the plausibility check. It is thereby made possible for a user of the blood-pressure measuring device to estimate whether the result of the blood pressure measurement can be used. It is also made possible for the user to take measures in order that the blood pressure can be measured correctly.

The display device is preferably a monitor. It is thereby possible to display the result of the plausibility check without providing a further device. Preferably, the result of the plausibility check is displayed in the form of colours. A positive result of a plausibility check is preferably displayed in green, a negative result of a plausibility check in orange. Preferably, the result of the plausibility check is displayed audibly. A negative result of a plausibility check is preferably displayed in the form of a warning tone, for example a rising and falling tone or a continuously repeating tone. A positive result of a plausibility check is preferably displayed by a quieter tone than a negative result of a plausibility check.

A result of a plausibility check thus permits the conclusion as to whether the system is functioning properly.

Preferably, the result of the plausibility check can be displayed in three stages, wherein the first stage comprises plausible results, the second stage deviating results and the third stage implausible results. It is thereby even more possible for a user of the blood-pressure measuring device to assess whether the blood-pressure measuring device, the rinsing device and/or the connection to a blood circuit are functioning correctly. Moreover, a user of the blood-pressure measuring device can be informed in this way, through the display of deviating results, that measures will shortly need to be taken.

Plausible results or positive results are results which permit the conclusion that the blood-pressure measuring device, the rinsing device and the connection to the blood circuit are functioning adequately Implausible results or negative results are mainly results which suggest a fault in the blood-pressure measuring device, the rinsing device and/or the connection to the blood circuit. Deviating results are mainly results which permit the conclusion that the blood-pressure measuring device, the rinsing device and the connection to a blood circuit are functioning properly, but also suggest that further plausibility checks would produce implausible results. Mainly, deviating results occur if there are deposits in the blood-pressure measuring device, in the rinsing device and/or in the connection to the blood circuit and there is an adequate rinsing.

Preferably, the computing device is set up to calculate the rinsing rate in the resistor taking into account a first measurement result which can be ascertained by the first pressure sensor and a second measurement result which can be ascertained by the second pressure sensor. It is thereby possible to easily ensure that the blood-pressure measuring device and the connections to the blood circuit are adequately rinsed.

The rinsing rate is preferably a value for the quantity of fluid per unit of time which passes through the resistor. Preferably, the rinsing rate is the volume of fluid which moves through a cross-section of the resistor within a period of time.

Preferably, the resistor is provided with an encoded version of the flow constant of the resistor. It is thereby easily possible to determine the flow constant of the resistor. Coding preferably serves to identify the resistor. Preferably, data are contained in the identification which are for the use of the resistor in the blood-pressure measuring device. Preferably, the code contains information as to what flow constant the resistor displays. Preferably, a resistor, a capacitor or a connector, particularly preferably a storage element or a chip, is used for coding.

The flow constant of the resistor is preferably a constant by which the rinsing rate can be calculated knowing the pressures in front of and behind the resistor.

Preferably, the computing device is set up to recognize the code. It is thereby easily possible to give the computing device access to the information contained within the code. Recognition of the code preferably comprises the conversion of the code into values, particularly preferably into a flow constant. Particularly preferably the recognition comprises the provision of values for calculations. Preferably, the encoder is set up such that it is connected briefly to the blood-pressure measuring device. It is thereby possible to save on lines.

Preferably, the resistor, the first pressure sensor and the second pressure sensor are arranged in a joint casing. It is thereby possible to provide the blood-pressure measuring device as a unit. This unit is preferably easily connectable to a rinsing device and to a connection to a blood circuit. Preferably, the blood-pressure measuring device is provided as a disposable device. It is thereby easily possible to provide a sterile blood-pressure measuring device. Preferably, the rinsing rate blood-pressure sensor, i.e. the resistor and the first and second pressure sensor, can be connected to any catheter.

Preferably, the resistor, the first pressure sensor and the second pressure sensor are integrated in a catheter. It is thereby particularly easily possible to use a blood-pressure measuring device. It is then not necessary for the user, preferably a doctor or care worker, to connect the blood-pressure measuring device to the catheter.

Preferably, the blood-pressure measuring device comprises a blood-sampling port. A contactless, protected (“closed”) blood-sampling or an intermittent rinsing of the catheter is thereby made possible.

Preferably, the blood-pressure measuring device comprises a cock which is set up to make possible a connection of the pressure sensors to the ambient pressure, wherein the cock can be operated mechanically, pneumatically, hydraulically and/or electrically. It is thereby possible in a particularly convenient way to set the pressure sensors to zero or to match them to the ambient pressure.

Preferably, at least two cocks are provided. Preferably, the cocks are set up to open and close at the same time. It is thereby possible to provide the cocks such that the pressure sensors are connected to the ambient pressure without components of the system between ambient pressure and pressure sensor making a setting of the pressure sensors to zero unnecessarily difficult. Because the cocks are set up to open and close at the same time, all pressure sensors can be set to zero at the same time and in a short period of time.

Preferably, the object is achieved by a method for carrying out a plausibility check on a blood-pressure measuring device comprising the steps: measuring a first pressure between a resistor and a rinsing device, measuring a second pressure between a resistor and a blood circuit, carrying out a plausibility check by comparing measurement results of the first pressure sensor and of the second pressure sensor.

Preferably, the plausibility check comprises: checking as to whether the pressure p1 measured by the first pressure sensor is lower than a predetermined maximum pressure p1max and/or checking as to whether the pressure p1 measured by the first pressure sensor is higher than a predetermined minimum pressure p1min and/or checking as to whether the difference between the pressure p1 measured by the first pressure sensor and the pressure p2 measured by the second pressure sensor is more than a predetermined minimum differential pressure DELTapmin and/or checking as to whether the difference between the pressure p1 measured by the first pressure sensor and the pressure p2 measured by the second pressure sensor is less than a predetermined maximum differential pressure DELTapmax.

Preferably, an alarm is sounded when the maximum pressure or the maximum differential pressure is exceeded or when the minimum pressure or the minimum differential pressure is not reached.

Preferably, the result of the plausibility check is displayed.

Preferably, the result of the plausibility check is displayed in three stages, wherein the first stage comprises plausible results, the second stage deviating results and the third stage implausible results.

Preferably, the rinsing rate in the resistor is calculated taking into account a first measurement result which can be ascertained by the first pressure sensor and a second measurement result which can be ascertained by the second pressure sensor.

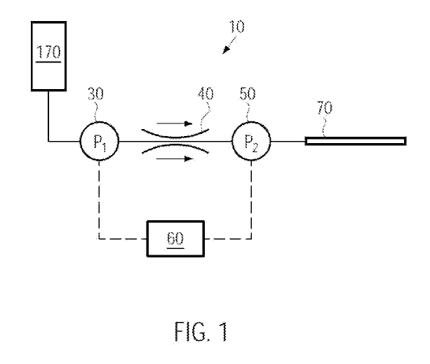

FIG. 1 shows a diagram of an embodiment example of a blood-pressure measuring device according to the invention 10. A bag 170 is connected to a first pressure sensor 30, the first pressure sensor 30 is connected to a resistor 40, the resistor 40 is connected to a second pressure sensor 50 and the second pressure sensor 50 is connected to a catheter 70. The first pressure sensor 30 and the second pressure sensor 50 each have a data line to a monitor 60.

The bag 170, the first pressure sensor 30, the resistor 40, the second pressure sensor 50 and the catheter 70 are connected to one another such that a fluid can flow from the 170 to the resistor 40 and from the resistor 40 to the catheter 70, wherein the pressure of the fluid before passing through the resistor 40 can be measured by the first pressure sensor 30 and after passing through the resistor 40 can be measured by the second pressure sensor 50. The first pressure sensor 30 and the second pressure sensor 50 are set up to pass the measured pressures to the computing device 60 via the data lines.

The computing device 60 is set up to check as to whether the first pressure measured by the first pressure sensor 30 is lower than a predetermined maximum pressure P1, whether the first pressure measured by the first pressure sensor is higher than a predetermined minimum pressure and whether the difference between the first pressure measured by the first pressure sensor and the second pressure measured by the second pressure sensor is more than a predetermined minimum differential pressure.

It can thereby be checked as to whether the blood pressure measured by the second pressure sensor 50 is plausible and whether the blood-pressure measuring device 10 is functioning properly. Should the bag 170 be empty, the first pressure measured by the first pressure sensor 30 will be lower than a predetermined maximum pressure. If there is a blockage in the blood-pressure measuring device 10, the first pressure measured by the first pressure sensor 30 will be higher than a predetermined minimum pressure. If an adequate flow of the fluid through the resistor 40 is not guaranteed, the difference between the first pressure measured by the first pressure sensor 30 and the second pressure measured by the second pressure sensor 50 will be less than the predetermined minimum differential pressure.

The use of false blood-pressure values to diagnose a patient can thereby be prevented. Deposits can also be prevented from forming in the catheter 70 and possibly leading, when detached, to an embolism, i.e. a partial or complete blocking of a blood vessel of a patient.

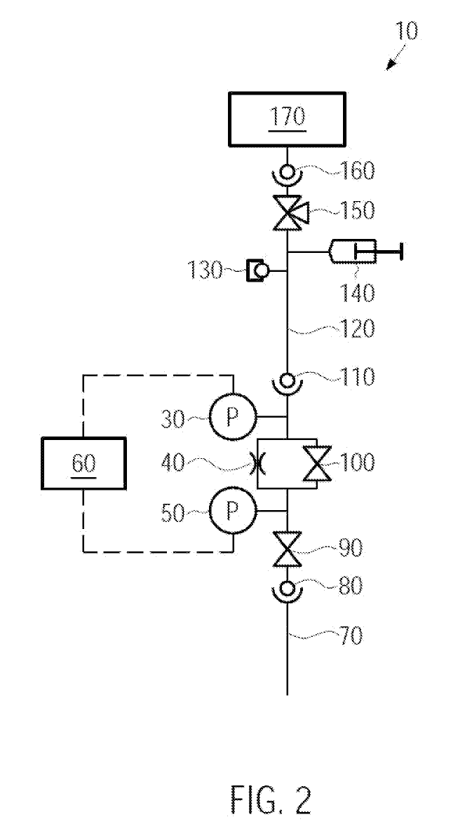

FIG. 2 shows a diagram of a further embodiment example of a second blood-pressure measuring device according to the invention 10. In addition to the blood-pressure measuring device according to the invention 10 shown in FIG. 1, a first coupling 80 to the catheter 70 is provided here. A first shutoff cock 90 is attached to this coupling 80. Furthermore, a second shutoff cock 100 is arranged parallel to the resistor 40. A second coupling 110 is provided on the side of the first pressure sensor 30 which points towards the bag 170. A blood-sampling port 130, a syringe 140 and a three-way cock 150 are provided on the side of the coupling 110 which points towards the bag 170. A third coupling 160 is arranged between the three-way cock 150 and the bag 170.

The zero points of the pressure sensors 30 and 50 can thereby easily be fixed. It is also possible to replace individual constituents of the system. For example, by loosening the first coupling 80 and the second coupling 110 the components first pressure sensor 30, resistor 40, second pressure sensor 50, second shutoff cock 100 and first shutoff cock 90 can be uncoupled from the blood-pressure measuring device 10 and replaced. By loosening the second coupling 110 and the third coupling 160 it is possible to uncouple the components blood-sampling port 130, syringe 140 and three-way cock 150 from the system and replace them with other components.

A blood sample can be taken by providing the blood-sampling port 130. Blood can be aspirated or the catheter 60 rinsed intermittently by providing the syringe 140.

To fix the zero point, the first shutoff cock 90 is closed, the second shutoff cock 100 opened and the three-way cock 150 opened so that ambient pressure applies at the first pressure sensor 30 and at the second pressure sensor 50.

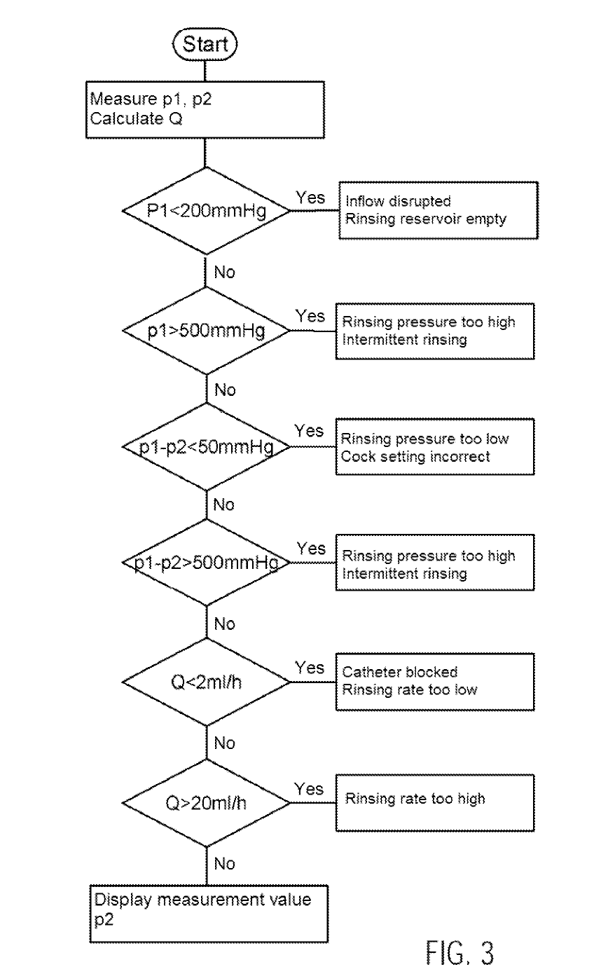

FIG. 3 shows a flowchart illustrating the course of a method according to the invention. At the start of a measurement, the pressures p1 and p2 are measured and the flow lying between calculated from the resistor reading. If p1 is lower than 200 mmHg, it is assumed that the inflow is disturbed, for example the rinsing reservoir is empty. If p1 is higher than 500 mmHg it is assumed that the rinsing pressure is too high and for example an intermittent rinsing has taken place. If the difference between p1 and p2 is less than 50 mmHg, the rinsing pressure is too low and for example the cock setting could be incorrect. If the difference between p1 and p2 is more than 500 mmHg, the rinsing pressure is too high, and here there may for example be an intermittent rinsing. If the flow is less than 2 ml per hour, it is assumed that the catheter is blocked and therefore the rinsing rate is too low. If the throughflow or the rinsing rate is greater than 20 ml per hour, the rinsing rate is too high. If all these plausibility observations are negative the measurement value p2 appears to be reliable and the measurement value p2 is displayed by the display device. Otherwise the measurement value is not displayed and the nature of the problem is shown.

### LIST OF REFERENCE NUMBERS

10 Blood-pressure measuring device
30 First pressure sensor
40 Resistor
50 Second pressure sensor
60 Computing device
70 Catheter
80 First coupling
90 First shutoff cock
100 Second shutoff cock
110 Second coupling
120 Tube
130 Blood-sampling port
140 Syringe
150 3-way cock
160 Third coupling
170 Bag

1-19. (canceled)

20. A blood-pressure measuring device comprising:
a resistor having a first end and a second end;
a first pressure sensor disposed on the first end of the resistor;
a second pressure sensor disposed on the second side of the resistor and configured for connection to a blood circuit, wherein the first pressure sensor, the resistor and the second pressure sensor are connected to each other in series;
a computing device connectable to the first pressure sensor and the second pressure sensor and configured to perform a plausibility check by comparing measurement results of the first pressure sensor and of the second pressure sensor.

21. The blood-pressure measuring device as recited in claim 20, wherein the plausibility check includes performing at least one of a first check as to whether a first pressure measured by the first pressure sensor is lower than a predetermined maximum pressure, a second check as to whether the first pressure is higher than a predetermined minimum pressure, a third check as to whether a difference between the first pressure and a second pressure measured by the second pressure sensor is more than a predetermined minimum differential pressure, and a fourth check as to whether the difference between the first pressure the second pressure is less than a predetermined maximum differential pressure.

22. The blood-pressure measuring device as recited in claim 21, further comprising an alarm device configured to sound an alarm when the maximum pressure or the maximum differential pressure is exceeded or when the minimum pressure or of the minimum differential pressure is not reached.

23. The blood-pressure measuring device as recited in claim 20, further comprising a display device configured to display the result of the plausibility check.

24. The blood-pressure measuring device as recited in claim 23, wherein the display device is configured to display the results in a first, a second and a third stage, wherein, the first stage includes plausible results, the second stage includes deviating results and the third stage includes implausible results.

25. The blood-pressure measuring device as recited in claim 20, wherein the computing device is configured to calculate a rinsing rate in the resistor taking into account a first measurement result ascertainable by the first pressure sensor and a second measurement result ascertainable by the second pressure sensor.

26. The blood-pressure measuring device as recited in claim 20, wherein the resistor is provided with an encoded version of a flow constant of the resistor.

27. The blood-pressure measuring device as recited in claim 26, wherein the computing device is configured to recognize the code.

28. The blood-pressure measuring device as recited in claim 20, further comprising a joint casing and wherein the resistor, the first pressure sensor and the second pressure sensor are disposed in the joint casing.

29. The blood-pressure measuring device as recited in claim 20, further comprising a catheter and wherein the resistor, the first pressure sensor and the second pressure sensor are integrated in the catheter.

30. The blood-pressure measuring device as recited in claim 20, further comprising a blood-sampling port.

31. The blood-pressure measuring device as recited in claim 20, further comprising at least one cock configured to enable a connection of the pressure sensors to ambient pressure, the cock can be operated in at least one of a mechanical, pneumatical, hydraulical and electrical manner.

32. The blood-pressure measuring device as recited in claim 31, wherien the at least one cock includes at least two cocks configured to open and close at the same time.

33. A method for performing a plausibility check on a blood-pressure measuring device having a resistor, a rinsing device, and a blood circuit, the method comprising:
measuring a first pressure between the resistor and the rinsing device;
measuring a second pressure between the resistor and the blood circuit,
comparing the first pressure and the second pressure.

34. The method as recited in claim 33, further comprising at least one of the following checks:
checking whether the first pressure is lower than a predetermined maximum pressure;
checking whether the first pressure is higher than a predetermined minimum pressure;
checking whether a difference between the first pressure the second pressure sensor is more than a predetermined minimum differential pressure; and
checking whether a difference between the first pressure and the second pressure is less than a predetermined maximum differential pressure.

35. The method as recited in claim 33, further comprising sounding an alarm when the maximum pressure or the maximum differential pressure is exceeded or when the minimum pressure or the minimum differential pressure is not reached.

36. The method as recited in claim 33, further comprising displaying result of the plausibility check.

37. The method as recited in claim 36, wherein the result of the plausibility check is displayed in a first stage, a second stage and a third stage, wherein the first stage comprises plausible results, the second stage deviating results and the third stage implausible results.

38. The method as recited in claim 33, further comprising calculating a rinsing rate in the resistor using the first pressure and the second pressure.
